(19) United States
(12) Patent Application Publication
(10) Pub. No.: US 2003/0050313 A1
(43) Pub. Date: Mar. 13, 2003

(54) METHOD FOR THE SIMPLIFIED PRODUCTION OF (3-CHLORO-4-FLOUROPHENYL)-[7-(3-MORPHOLIN-4-YL-PROPOXY)-6-NITRO-QUINAZOLINE-4YL]-AMINE OR (3-CHLORO-4-FLUOROPHENYL)-[7-(3-MORPHOLIN-4-YL-PROPOXY)-6-AMINO-QUINAZOLINE-4-YL]-AMINE

(76) Inventors: Hubert Barth, Emmendingen (DE); Klaus Steiner, Emmendingen (DE); Simon Schneider, Emmendingen (DE)

Correspondence Address: Rosanne Goodman, Warner Lambert Company, 2800 Plymouth Road, Ann Arbor, MI, 48105, US

(21) Appl. No.: 10/204,911

(22) PCT Filed: Jan. 23, 2001
(86) PCT No.: PCT/EP01/00695

(30) Foreign Application Priority Data
Feb. 26, 2000 (DE) 100 09 267.5

(51) Int. Cl. A61K 31/5377; C07D 413/02

(52) U.S. Cl. 514/233.8; 544/116

(57) ABSTRACT

The invention concerns a one-pot reaction for the preparation of (3-chloro-4-fluorophenyl)-[7-(3-morpholin-4-yl-propoxy)-6-nitroquinazolin-4-yl]-amine (I)

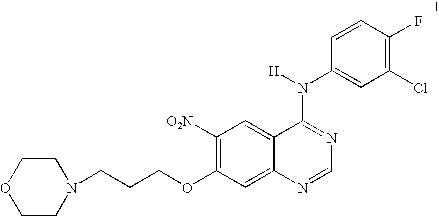

or of (3-chloro-4-fluorophenyl)-[7-(3-morpholino-4-yl-propoxy)-6-aminoquinazolin-4-yl]-amine (VII)

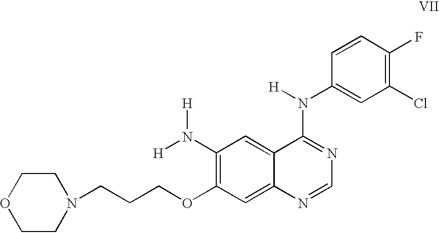

In the case of (3-chloro-4-fluorophenyl)-[7-(3-morpholin-4-ylpropoxy)-6-nitroquinazolin-4-yl]-amine (I)

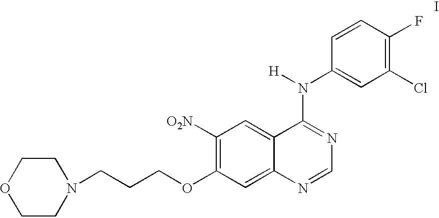

it is a question of a key compound for the preparation of N-[4-[(3-chloro-4-fluorophenyl)-amino]-7-[3-(4-morpholinyl)-propoxy]-quinazolinyl]-propenamide dihydrochloride (II)

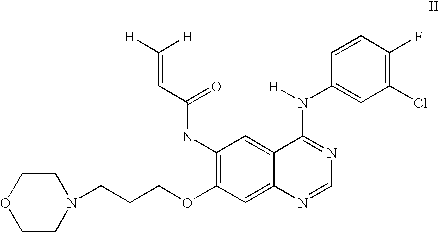

a representative of a new class of highly effective irreversible tyrosine kinase inhibitors of the EGFR (epidermal growth factor receptor) which is to be used for the treatment of various tumours, which is described, for example, in WO 97/38983.

In the case of the above-mentioned irreversible tyrosine kinase inhibitors of EGFR, it is a question of substituted 4-anilinoquinazoline derivatives which hitherto could, in part, only be prepared in an up to 12 stage synthesis. The original synthesis steps are described in J. Med. Chem. 1996, 39, 918-928 and WO 97/38983.

Starting material for the previous synthesis was isomerically pure 7-fluoro-6-nitroquinazolin-4(3H)-one (III)

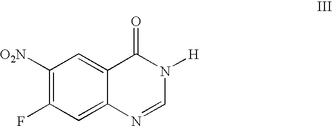

which was reacted without solvent with a 55 molar excess of thienyl chloride with the addition of catalytic amounts of DMF to give 4-chloro-7-fluoro-6-nitroquinazoline (IV)

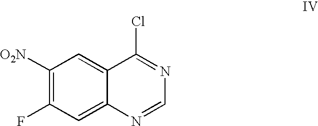

After distilling off of the thienyl chloride, the 4-chloro-7-fluoro-6-nitroquinazoline obtained as crude product was reacted portionwise with a solution of 1 equivalent of 3-chloro-4-fluoroaniline and 2 equivalents of the highly toxic N,N-dimethylaniline in 2-propanol. After 6 hours stirring at 25° C., after an aqueous working up there was obtained 4-(3-chloro-5-fluoroanilino)-7-fluoro-6-nitroquinazoline (V)

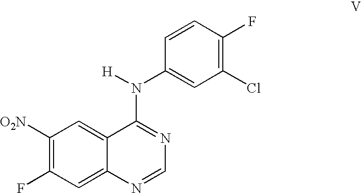

in about 90% yield as crude product.

To a suspension of 1 equivalent of 4-(3-chloro-4-fluoroanilino)-7-fluoro-6-nitroquinazoline (V) and 1.5 equivalents of 3-(4-morpholino)-propan-1-ol (VI)

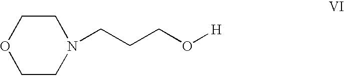

in dimethyl sulphoxide (DMSO) was added dropwise a solution of 3 equivalents potassium trimethyl silanoate in DMSO and the reaction mixture stirred for about 6 hours. After an aqueous working up, (3-chloro-4-fluoro-phenyl)-[7-(3-morpholin-4-yl-propoxy)-6-nitroquinazolin-4yl]-amine (I) was obtained in about 89% yield.

This synthesis step proved to be especially problematical since, in the case of batch enlargement, there were obtained only very variable yields and, in part, also clearly smaller yields than described.

Reactions of 4-(3-chloro-4-fluoroanilino)-7-fluoro-6-nitroquinazoline (V) and 3-(4-morpholino)-propan-1-ol (VI) and solid sodium hydride in THF analogously to the process described in WO 97/38983 also lead to only unsatisfactory results.

The (3-chloro-4-fluorophenyl)-[7-(3-morpholin-4-yl-propoxy)-6-nitroquinazolin-4-yl]-amine (I) obtained was subsequently hydrogenated inter alia over Raney nickel in THF as solvent to give (3-chloro-4-fluorophenyl)-[7-(3-morpholin-4-yl-propoxy)-6-aminoquinazolin4-yl]-amine (VII)

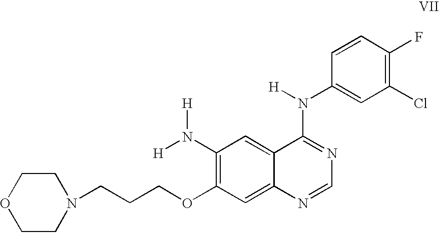

and then further reacted to give (II) or its dihydrochloride trihydrate.

Therefore, it was the task of the invention to develop an economic and technically practicable process for the preparation of the above-mentioned key compounds (I) or (VII). Hereby should:

1.) the amount of the thionyl chloride used be reduced,

2.) the use of the highly toxic N,N-dimethylaniline be avoided,

3.) DMSO replaced by a cheaper solvent,

4.) potassium trimethylsilanoate or sodium hydride be replaced,

5.) the yields of the reaction of (I) and (VI) be constantly good,

6.) and, in each case, an isolation of the individual compounds (IV), (V) and possibly of (I) be omitted.

Therefore, the subject of the invention is the combination of the individual reaction steps to give a one-pot reaction.

Surprisingly, it was found that the chlorination reaction of (III) with thionyl chloride to give (IV), the reaction of the chloride (IV) formed with 3-chloro-4-fluoroaniline to give (V) and the subsequent reaction of (V) with 3-morpholin-4-yl-propan-1-ol (VI) to the key compound (I) in outstanding yield can be combined, without isolation of the intermediate compounds to a one-pot reaction with 3 reaction steps, as illustrated in the reaction scheme (FIG. 1).

Furthermore, it was, surprisingly, found that it is also not necessary to isolate the compound (I) but rather that the reaction mixture obtained in the case of the above-mentioned one-pot reaction can be used directly for the hydrogenation to give (VII), i.e. to a one-pot reaction with 4 reaction steps.

Furthermore, in the case of the chlorination reaction, the 55 molar excess of thionyl chloride can be reduced to an 11.5 molar excess. After distilling off of the thionyl chloride, residual thionyl chloride is distilled off azeotropically several times with toluene. In the case of the last toluene distillation, the toluene must only be distilled off to the extent that a still readily stirrable coarsely crystalline residue remains behind. The so-formed chloride (IV) is very pure and is mixed directly with a tetrahydrofuran/tert.-butanol mixture (7:3). For the further course of the reaction, the use of this tetrahydrofuran/tert.-butanol mixture is of great importance. Surprisingly, it was found that the tert.-butyl alcohol added to the reaction mixture catalyses the desired substitution reaction.

In the following reaction of the chloride (IV) with 3-chloro-4-fluoroaniline, the highly toxic N,N-dimethyl-aniline initially used as acid acceptor could be replaced by the amine necessary in the 3rd reaction step, 3-morpholin-4-yl-propan-1-ol (VI). After about 24 hours stirring at room temperature, the chloride (IV) was completely reacted to give the aniline derivative (V). Surprisingly, in the case of this reaction, the alcohol group of the added base does not react. The hereby resulting yellow to orange coloured suspension was mixed directly with a potassium tert.-butylate/THF solution. The 3-morpholin-4-yl-propan-1-ol (VI) used as acid acceptor in the previous step is again converted into the free base which then, in the presence of potassium tert.-butylate, further reacts immediately in the desired way with the aniline derivative (VI) already present in the reaction mixture to give the desired (I) which, after quenching with an ice/ethanol/hydrochloric acid mixture, was obtained in a surprisingly good total yield of about 95% with an also surprisingly good purity of about >98%.

This very good course of the reaction was the more surprising since the tert.-butyl alcohol present in the reaction mixture and also newly forming does not react in analogous manner as 3-morpholin-4-yl-propan-1-ol (VI) with (V) to give the corresponding tert.-butyl ether.

Furthermore, it was surprising that the NaH used for such substitution reactions (WO 97/38983; J. Med. Chem. 35, 14, 1992, 2617-2626; J. Am. Chem. Soc. 76, 1954, 3032; Heterocycles 22, 1, 1984, 73-78), sodium amide (J. Org. Chem. 59, 21, 1994, 6194-6199) or potassium trimethylsilonate could be replaced by the non-problematically handlable potassium tert.-butylate.

Surprisingly, the product (I) obtained in the reaction mixture was of such a high purity that the reaction solution could be used directly for the subsequent hydrogenation without isolation of (I).

### EXAMPLE 1

One-Pot Reaction for the Preparation of (3-chloro-4-fluorophenyl)-[7-(3-morpholin-4-yl-propoxy)-6-nitroquinazolin-4-yl]-amine (I).

150 g 7-fluoro-6-nitroquinazolin-4(3H)-one (III) are suspended in 600 ml thionyl chloride and, after addition of 6 ml DMF, boiled under reflux for 24 hrs. A clear solution hereby results. About 350 ml thionyl chloride are distilled off in a vacuum. The resulting coarsely crystalline suspension is mixed with about 600 ml toluene. About 800 ml are distilled off in a vacuum. This distillation was further repeated 3 times with, in each cape, 600 ml fresh toluene. In the case of the last distillation, the toluene is distilled off as far as possible. There results a coarsely crystalline suspension which, at all times, remains well stirrable.

The almost dry residue is mixed with 1.2 l of a tetrahydrofuran/tert.-butanol mixture (7:3). The resulting suspension is cooled to about 10° C. With good stirring and cooling, a solution of 114 g 3-chloro-4-fluoroaniline and 258 g 3-morpholin-4-yl-propan-1-ol (VI) in 300 ml THF/tert.-butanol (7:3) is so added dropwise over the course of about 20 min. that the temperature in the reactor remains between 10° C. and 15° C. The initially yellowish suspension becomes thinner and colours orange in the course of the dropping in.

One allows the reaction mixture slowly to come to room temperature and subsequently stirs at room temperature for at least 24 hrs.

To the yellow-orange suspension is so added dropwise, with good stirring and gentle cooling during the course of about 20 min., a solution of 324 g potassium tert.-butylate in 1.86 l tetrahydrofuran that the temperature in the reactor remains between 15° C. and 20° C. After addition of about ⅓ of the potassium tert.-butylate/THF solution, the whole reaction mixture becomes dark red coloured.

After about 30 minutes after-stirring, the mixture is immediately stirred into a mixture of 5.4 kg ice, 6.0 l ethanol and 1.8 l hydrochloric acid (pH of the solution about 8). There hereby first results a yellow-orange solution. After brief stirring, a yellow product crystallises out. The resulting suspension is after-stirred for about 5 hrs at about 0° C. and subsequently filtered off with suction. The filter cake is after-washed twice with, in each case, 500 ml ice-cold ethanol.

The product is pre-dried in a circulating air cabinet first at 40° C. and subsequently at 60° C. to constant weight (yield: 316.5 g=95.5%; HPLC purity: 98.48 rel. %; H₂O (K.F.) 3.69% m.p. 257° C.

### EXAMPLE 2

One-Pot Reaction for the Preparation of (3-chloro-4-fluorophenyl)-[7-(3-morpholin-4-yl-propoxy)-6-aminoquinazolin-4-yl]-amine (VII).

20 g 7-fluoro-6-nitroquinazolin-4-(3H)-one (III) are suspended in 80 ml thionyl chloride and, after addition of 20 drops of DMF, boiled under reflux for 24 hrs. A clear solution hereby results. About 60 ml thionyl chloride are distilled off in a vacuum. The resulting coarsely crystalline suspension is mixed with about 60 ml toluene. About 60 ml are distilled off in a vacuum. This distillation is repeated 3 times with, in each case, 60 ml fresh toluene. In the last distillation, the toluene is distilled off as far as possible. There results a coarsely crystalline suspension which at all times remains well stirrable.

The almost dry residue is mixed with 160 ml of a tetrahydrofuran/tert.-butanol mixture. The resulting suspension is cooled to about 10° C. With good stirring and cooling, a solution of 15.2 g 3-chloro-4-fluoroaniline and 34.4 g 3-morpholin-4-yl-propan-1-ol (VI) in 40 ml THF/tert.-butanol (7:3) is so added dropwise over the course of about 20 min. that the temperature in the reactor remains between 10° C. and 15° C. The initially yellow suspension becomes thinner in the case of the dropping in and colours towards orange.

One allows the reaction mixture to come slowly to room temperature and subsequently stirs for at least 24 hrs. at room temperature.

To the yellow-orange suspension is so added dropwise, with good stirring and gentle cooling over the course of about 20 min., a solution of 43.2 g potassium tert.-butylate in 250 ml tetrahydrofuran that the temperature in the reactor remains between 15° C. and 20° C. After addition of about ⅓ of the potassium tert.-butylate/THF solution, the whole reaction mixture becomes dark red coloured.

After about 30 minutes after-stirring, the reaction mixture is mixed at 0° C.-5° C. with a mixture of 20 ml hydrochloric acid and 30 ml water and diluted with a further 200 ml THF. After 20 minutes stirring in an ice bath, the reaction mixture is filtered clear over 50 g Celite. The filter cake is washed out with 100 ml THF. The filtrate is mixed with 31 g Raney nickel and hydrogenated at room temperature for 3 hrs. at 3.5 bar with hydrogen. After filtering off the catalyst with suction, the filtrate is evaporated to dryness and the residue stirred with 80 ml ethanol at about 2° C. The precipitated product is filtered off with suction and washed with a little cold ethanol. After drying in a circulating air drying cabinet at 60° C., there are obtained 32.1 g (77.7%) of product.

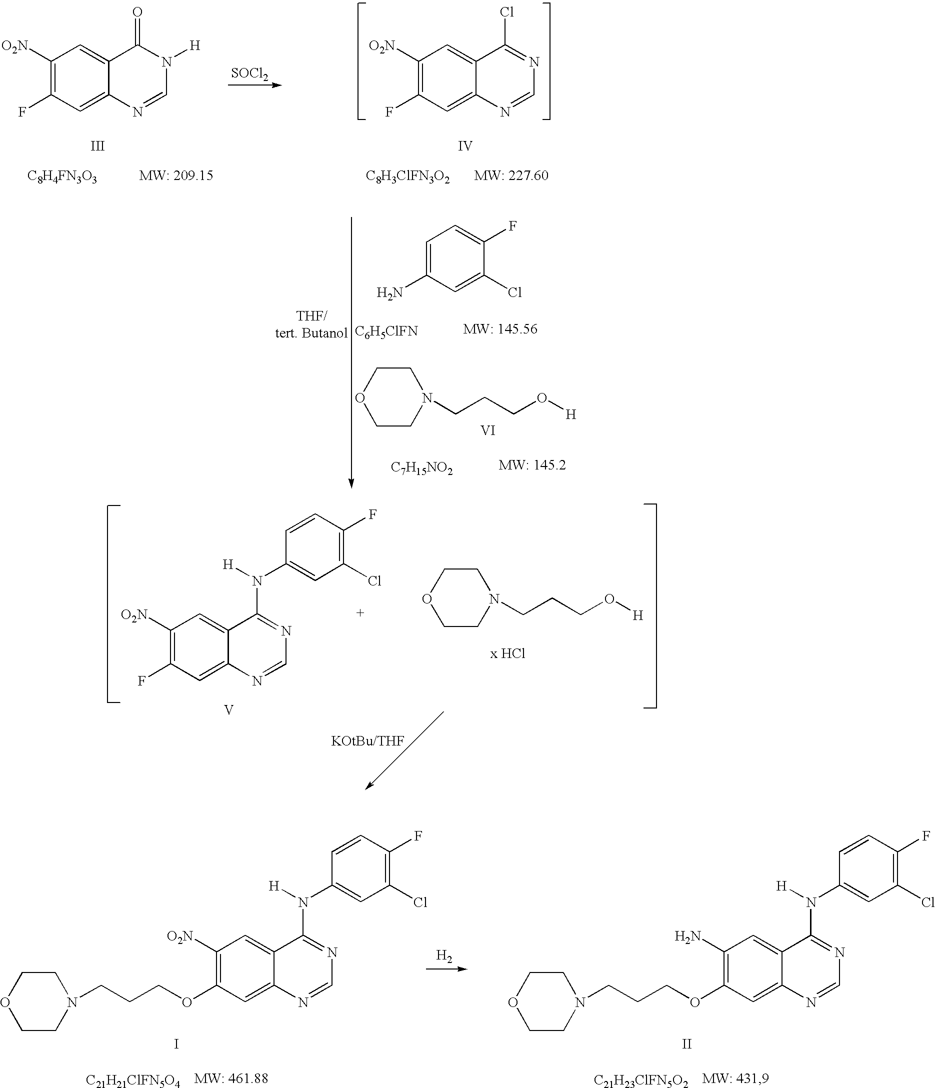

1. Process for the preparation of (3-chloro-4-fluorophenyl)-[7-(3-morpholin-4-yl-propoxy)-6-nitroquinazolin-4-yl]-amine (I) 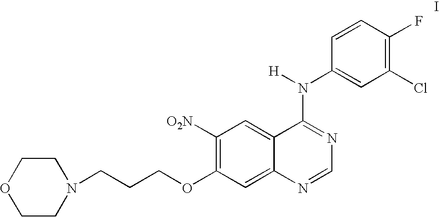
or of (3-chloro-4-fluorophenyl)-[7-(3-morpholin-4-yl-propoxy)-6-aminoquinazolin-4-yl]-amine (VII) 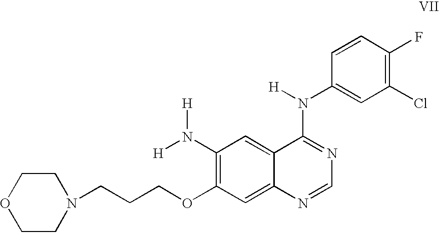
characterised in that, in a one-pot reaction, in 3 or 4 reaction steps, one first reacts 7-fluoro-6-nitroquinazolin-4(3H)-one (III) 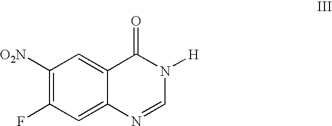
with thionyl chloride to give 4-chloro-7-fluoro-6-nitroquinazoline (IV) 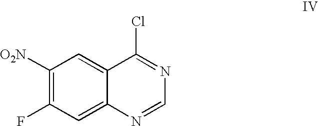
reacts this with 3-chloro-4-fluoroaniline to give 4-(3-chloro-4-fluoroanilino)-7-fluoro-6-nitroquinazoline (V) 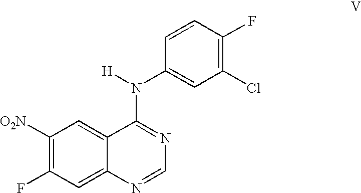
and subsequently with 3-morpholin-4-yl-propan-1-ol (VI) 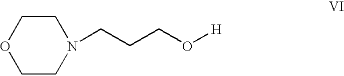
to give (I) and possibly hydrogenates (I) directly in the reaction solution to give (III).
